(19) United States
(12) Patent Application Publication
(10) Pub. No.: US 2021/0342344 A1
(43) Pub. Date: Nov. 4, 2021

(54) Weighed Order Decision Making with Visual Representation

(71) Applicant: NoviSystems, Inc., Garner, NC (US)

(72) Inventors: Michael Kowolenko, Garner, NC (US); John C. Bass, Garner, NC (US); Meaghan E. Johnson, Garner, NC (US); Andrew Brown, Garner, NC (US); Michael S. Brown, Garner, NC (US); Jesse Simpson, Garner, NC (US)

(21) Appl. No.: 17/369,336

(22) Filed: Jul. 7, 2021

Related U.S. Application Data
(63) Continuation-in-part of application No. 16/453,805, filed on Jun. 26, 2019.

(51) Int. Cl. G06F 16/2458; G06F 16/2457; G06N 20/00; G06F 3/0482; G06F 3/0484

(52) U.S. Cl. CPC G06F 16/2465; G06F 16/24578; G16H 10/60; G06F 3/0482; G06F 3/0484; G06N 20/00

(57) ABSTRACT

A system for the dynamic analysis of unstructured data where feedback loops exist between the user and the machine resulting in improved specificity and content (accuracy and precision) with regard to the results obtained from the machine learning algorithms. A Graphic User Interface (GUI) controls the configuration and deployment of all the features of the Intelligence Augmentation System (IAS) including data capture and processing, analytics, and feedback. Results of one set of algorithms can be forwarded to subsequent tools with the system for further analysis and planning using decision algorithms. The results are configured using a GUI that can manipulate the data in dynamically, allowing immediate visualization of user queries.

### CLAIM TO PRIORITY

This application claims under 35 U.S.C. § 120, the benefit of the application Ser. No. 16/453,805, filed Jun. 26, 2019, titled “Intelligence Augmentation System for Data Analysis and Decision Making” which is hereby incorporated by reference in its entirety.

### COPYRIGHT NOTICE

A portion of the disclosure of this patent document contains material which is subject to copyright protection. The copyright owner has no objection to the facsimile reproduction of the patent document or the patent disclosure, as it appears in the Patent and Trademark Office patent file or records, but otherwise reserves all copyrights whatsoever.

### BACKGROUND

Data Mining is the process of extracting insight from large amounts of structured data where features have been predefined. This type of data is often found in databases and collections of databases (e.g., data warehouses). Textual or unstructured data such as free formed text where features are derived by the reader familiar with the content and context of the words written in documents can be mined for content classification or fact extraction. Unfortunately, many software systems for analytics and machine learning focus on specific domains. The challenge is designing a system that can be used by business users with little experience in data sciences to extract relevant information and perform analysis and visualization of the results.

Unstructured text data mining is often used by business intelligence organizations to capture public perceptions regarding products, events, etc. It has been used in healthcare to extract information from electronic medical records, and in law enforcement to extract information regarding crimes.

Information systems are created through the use of APIs and other programming structures to upload, manage, maintain, and update information provided to a user. The user attaches to and interacts with the data display through a graphical user interface that serves as the front end and user experience for a user. Information is often presented to a user in the form of a user dashboard that presents information to a user in a digestible format based upon the requirements of a user. Modification and update of the information displayed and the manner of display requires programming efforts by the creator of the information system.

Historically, data is often fed to a user dashboard for the consumption of the user, but there is typically little to no recommendation from the system for the user in how to consume or utilize the information presented. More recent systems have begun to imbue user dashboard creation algorithms with some derived preference and usability analysis based upon the interaction of a user with the information presented in the user dashboard display.

### BRIEF DESCRIPTION OF THE DRAWINGS

Certain illustrative embodiments illustrating organization and method of operation, together with objects and advantages may be best understood by reference to the detailed description that follows taken in conjunction with the accompanying drawings in which:

FIG. 1 is a view of an Intelligence Augmentation System (IAS) features consistent with certain embodiments of the present invention.

FIG. 2 is a view of the IAS system configuration consistent with certain embodiments of the present invention.

FIG. 3 is a flow diagram for data import into the system consistent with certain embodiments of the present invention.

FIG. 4 is a flow diagram for word tokenization and analysis consistent with certain embodiments of the present invention.

FIG. 5 is a view of a machine language functionality processing capability consistent with certain embodiments of the present invention.

FIG. 6 is a view of a machine language parameter input process consistent with certain embodiments of the present invention.

FIG. 7 is a view of a process for the creation of a new corpus definition consistent with certain embodiments of the present invention.

FIG. 8 is a view of a machine language analysis process consistent with certain embodiments of the present invention.

FIG. 9 is a view of a process for performing training of a machine language analysis capability consistent with certain embodiments of the present invention.

FIG. 10 is a view of a process for the creation of a knowledge graph consistent with certain embodiments of the present invention.

FIG. 11 is a view of a process for weighted order decision making consistent with certain embodiments of the present invention.

### DETAILED DESCRIPTION

While this invention is susceptible of embodiment in many different forms, there is shown in the drawings and will herein be described in detail specific embodiments, with the understanding that the present disclosure of such embodiments is to be considered as an example of the principles and not intended to limit the invention to the specific embodiments shown and described. In the description below, like reference numerals are used to describe the same, similar or corresponding parts in the several views of the drawings.

The terms “a” or “an”, as used herein, are defined as one or more than one. The term “plurality”, as used herein, is defined as two or more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

Reference throughout this document to “one embodiment”, “certain embodiments”, “an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

Data is considered to be a set of values of subjects in a digital format that is storable and transmissible by computer systems.

The database is an ordered collection of data stored in a digital format on a computer system. Databases are maintained by database management systems (DBMSes). Queries to some databases are codified in the Structured Query Language (SQL).

The programming language is a formal language which comprises a set of instructions that produce various kinds of output. Programming languages are used in computer programming to implement algorithms.

The operating environment is composed of the operating system, communications software, software utilities, and platform software necessary for users to run application software.

The computer system is a set of devices that execute computational operations, store data used for input to computational operations and which are generated from computational operations, and transmit and receive data to and from other computer systems.

The use of lemma in this document refers to a heading indicating the subject or argument of a literary composition, an annotation, or a dictionary entry.

The use of Machine Learning (ML) in this document refers to one or more learning systems capable of identifying and processing fields in unknown input data to classify and predict the future state of the input data upon being trained in the definition and analysis of one or more training data sets by one or more human users.

The Healthcare Decision Platform (HDP) system is an integrated system for extracting information from healthcare related systems necessary for decision making. The system, a series of software algorithms that receives input from the user via a graphical user interface GUI resulting in the aggregation of information data fusion using tools such as natural language processing NLP that can be analyzed for relationships or classifications of relationships by machine learning algorithms.

In an embodiment, many analytical applications have the capability of analyzing aggregate views of data but are unable to perform analytics requiring real time join functions between different data tables and allow the user to see the results of analysis under these dynamic conditions. The opportunities in “Big Data” are the fusion of these data sets, however most database systems require complex join functions and extensive understanding of structured query language (SQL) to derive analytics and insights from the aggregate data views.

In the embodiment herein described, if the system can extract text from any business records management system and apply natural language processing NLP to the output, it can assist in multiple business processes including review of decision recommendation and justification.

In addition, the system of text extraction can be coupled with NLP to search medical literature such as PubMed® National Center of Biolnformatics (NCBI) to provide data regarding the current standard of care regarding a given diagnosis. This information can then be used to justify the treatment of the patient.

Using data fusion of data from electronic medical records, the data regarding staffing numbers, qualification, and training obtained from the ERP system along with historian data from hospital facilities systems, the quality of care of the patients can be assessed by viewing outcomes based on the integration of these factors. For example, patients clustered by a given disease type and socioeconomic factors, given specific training by one individual have a better outcome than given training by a different individual provides an opportunity to address training programs.

Finally, because the systems' rules-based system can be easily configured, medical staff can easily configure text analytics processes to extract facts from medical records making the identification of patients with defined signs and symptoms straightforward to isolate. Once isolated, quantitative data associated with the patient can be correlated with factors such as outcome, drug treatment, etc. using the built-in machine learning algorithms.

Unstructured text data mining is often used by business intelligence organizations to capture public perceptions regarding products, events, etc. by analyzing textual data input to the system. In non-limiting examples, such text data mining has been used in healthcare to extract information from electronic medical records, and in law enforcement to extract information regarding crimes. The challenge of Unstructured Text Analytics in data mining of text is the ambiguous nature of language. Each domain such as healthcare or crime requires intensive input from the subject matter expert (SME) in order to be effective. An SME may develop the lexicon required by the machine to perform the data mining task on unstructured data.

In an embodiment, the Healthcare Decision Platform HDP comprises 4 major components: Ingestion of data, formation of a common data tables, integrated analytics including NLP and machine learning, and a user configurable interactive Dashboard that can display or process data for further analytics and display. Many of the components have been described in patent application “An Intelligence Augmentation System for Data Analysis and Decision Making” Docket ID: NOV-npr-001, which is included by reference herein in its entirety.

In an embodiment, the IAS comprises six major components. The first of these encompasses the data capture for use by the system. Data exists in many formats, such as text documents (multiple formats; xdoc, txt, csv, html, web crawls) binary files (PDF), or in structured data formats (databases, xml,) that enumerate relationships between data fields and elements. In the IAS system, data stored on local networks or available on the web can be accessed by the IAS system when proper communications are established and data access is by default, such as publicly available data or open data access, or data access is granted by the owner of the data. The data connector to establish the communication and access the required data is built into the system and uses the appropriate database connectors for relational databases and additional pre-configured data connectors for other data types. The system when deployed is configured so that network system administrators provide access to databases, data stores, and file systems.

In an embodiment, for text analysis Data Tables stored in a Data Store can undergo the analysis of input text through the process of text analytics. The intent of text analytics is to extract facts from textual data or to classify text as meeting conditions defined by the user. Text, unlike quantitative data, has a high degree of ambiguity because of the contextual meaning of words. The innovation set forth in this document describes a process where users “seed” the dictionaries with a set of terms, the system compares the terms to a thesaurus, extracts sentences from the corpus of documents and requests feedback. In addition, the system uses machine learning algorithms to supplement the thesaurus resulting in improved specificity and context with relatively low SME input. To improve context and specificity, the integrated system text tool integrates data preparation, novel approaches to dictionary supplementation, and machine learning to provide contextually relevant fact extraction and classification of documents.

Selection of Natural Language Processing on the home page provides the functionality for implementing Natural Language Processing. Natural Language Process workflow offers the user two choices, a rules-based system using dictionaries, or machine learning. In a rules-based system, the system is directed by the user to annotate the document using the dictionaries developed using the Dictionary Editor. The advantage of a rules-based system is that the system will only annotate what has been defined as a term of interest, this term of interest becomes a dictionary term.

In a non-limiting example, to overcome the need for programmers to develop the code necessary for performing the task of annotation, the users are directed to a Dictionary Matrix Table where a data table with its respective fields may be displayed as rows, while each dictionary is displayed as a column. The user simply selects which dictionaries should be matched with which fields. The selection process has the option to be global (all dictionaries, all columns). Following the selection process, the annotation process is initiated and the machine annotates the data in the data table. Output is an index associated with the data table stored in a data store.

The second feature is the intelligence augmentation system deployed for utilizing machine learning. The IAS provides a multifaceted approach to utilizing machine learning that makes use of a feedback loop based on a rules-based system to improve the specificity and context of returns generated by the machine learning algorithms. The concept is that the use of dictionaries supplemented with the thesaurus feedback tool isolates facts and/or content of relevance. The identified facts and/or content become the training data for the machine learning algorithms.

The system generation of training data can be a tedious, time consuming process requiring manual annotation of documents. To overcome this issue, the system utilizes the output from a rules-based system coupled with part of speech (POS) analysis to generate phrases that have the appropriate specificity and context for the domain under investigation. The dictionaries provide the specificity, use of POS improves context as placement of terms in noun-verb-noun relationships uses rules of grammar to improve the relevancy of the terms that are used as either positive or negative training data in the machine learning models. These activities are performed on specific fields selected from the cleaned text, where cleaned text consists of known text fields and known contextual references for the text fields.

In an embodiment, the machine-learning learning system included with the IAS provides the user with information concerning topics that were not readily apparent to the user. In a non-limiting example, if the user developed dictionaries that isolated phrases that contained information concerning demographics and purchases, the rules-based system may retrieve facts such as “single males that purchase skateboards” if the noun for the verb purchase was restricted to skateboard and skateboarding items. The machine learning model may return a list of potential purchase items including skateboards but would expand that list to possible items contained in the documents such as cars, music, etc. that may be contextually relevant to those individuals that have historically purchased skateboards. The user can then request that one or more of the newly presented potential purchase items be added to the data table.

The text tools deployed with the IAS enable the user to develop models for fact extraction and text classification without a deep understanding of programming. The system relies on the user's expertise in the field to initiate the process and provide feedback to develop models for data extraction and text classification. The system is vertical agnostic and can be used by any subject matter expert.

In an embodiment, the IAS can perform classification and prediction calculations of user data through instantiating a series of algorithms that may be provided inputs generated by the preprocessing routines. The preprocessing routines receive input from a feedback system consisting of a user interface, the data under investigation, and the aforementioned routines. In addition, the system must be informed if the data model required is supervised or unsupervised learning. The user is prompted to characterize the query. Once filtering is complete and data visualized, the filtered data can be sent to directly to the machine learning algorithms.

This user input allows the IAS to select the appropriate set of machine learning algorithms to apply to the problem. The data is organized as a series of columns. The selection of a column represents the value a user wants to classify and/or predict without showing how the other data columns or features contribute to the analysis/prediction. This data isolation leads to the application of supervised learning algorithms where a selection of one column of data while requesting data grouping in an attempt to cluster data “likes”, where a “like” may be a similarity between two fields or data groups that permits the analysis of data to be performed more efficiently, may direct the system to supervised or unsupervised learning algorithms to optimize the processing of the data without requiring programmer intervention.

In an embodiment, the IAS has a GUI that allows non-programmers to develop queries of structured and unstructured data processed by the IAS algorithms.

The system employs a user interface to direct the user to add data analysis functions called widgets to the display using simple drag and drop user interface cues.

The configuration of the data display is referred to as a dashboard. Each dashboard is associated with a primary data table in the data store. During the data import process, the system may automatically import key relationships that exist in database tables and the system may allow the user to define new relationships in data tables imported into the IAS. Automatically importing key relationships increases the user's ability to define relationships between data sets without the need of a programmer.

In an embodiment, the system has the ability to generate knowledge graphs through the use of the dashboard application. Knowledge Graphs are useful in the visualization of relationships between entities. The Knowledge Graphs can also display distance relationships between entities. In a non-limiting example, the system uses the ability of NoviLens, a natural language processing capability native to the IAS, to filter data through the NLP annotation process and Machine Learning algorithms that may provide the data tables for the widgets. This function takes the filtered results and via a user interface, prompts the user for relationships between features.

In an embodiment, the user may select a filtering function for the data displayed in a dashboard based upon a “widget” query, where the “widget” may be any predefined filter requested by the user. To avoid any requirement for programming assistance, through a series of drop-down menus, the user may select the relationships that are to be established. The first is the Primary Node, or the central feature, that is the initiation point of the relationships to be established. The user may then select the adjacent feature through another dropdown menu. These two features, the central feature and the adjacent feature, need to be linked by a relationship in the data table. This relationship is the edge value, selected as another column from the data table. The result, as displayed to a user, is a visualized graph of the relationship between the various features selected. This visualized graph may, in turn, be further filtered via a query widget.

In an embodiment, the objective extraction and analysis of facts addresses many of the activities required by business analysts. However, there is a need for a somewhat subjective methodology in determining prioritization of decision making. In a non-limiting example, the decision on what automobile to buy may be driven by different priorities depending on the purchaser. A family of six has different requirements than a single person with regard to seating capabilities. A framework to manage these decision priorities has been built into the IAS system. This model uses the NLP and filtering capabilities of the IAS to collect and isolate the necessary facts. The IAS may then apply a series of weighted order decision algorithms to the data. Another unique feature is the user interface that allows the user to determine categories and scores as well as weights, then run “what if scenarios” to determine how changing preferences can change outcomes.

In an embodiment, to overcome the need for programmers to develop the code necessary for performing the task of annotation, the users may open a Directory Matrix Table where a data table with its respective fields may be displayed as rows, while each dictionary is displayed as a column. The user simply selects which dictionaries should be matched with which fields. The selection process has the option to be global, connecting with all dictionaries, and all columns. Following the selection process, the annotation process is initiated and the machine annotates the data in the data table. Output is an index associated with the data table stored in a data store.

In an embodiment, rules-based systems may not be based on statistics, but, rather, on token matching. In this case, the dictionary term is the token. The compute function is the matching of the token present in the dictionary with the presence of the token in the input data, the function initiated by selecting Searches.

Briefly, the user selects the patient record using the FHIR importer. The selected patient record is then processed by the pipeline into sentences and tokens for use by a token or term finder. Once entering the process, the text is evaluated for the presence of anaphoras. If present, the sentence is discarded. The next step is for the sentence to be categorized as being associated with male or female based on text tokens.

The sentence and its label are compared to definitions for terms that have been pre-defined and/or pre-configured in the system data tables, if there are matches, the sentence is scored. If there is no match of the descriptors between the sentence and the determined definition, the sentence is compared to MESH terms that are cross referenced with data table definitions that are exterior to, but accessible by, the system. These are viewed as synonyms. Recommendations for coding choices are now based on the synonym values.

The system generates a specificity score based on the number of descriptors and modifiers present in the analyzed text record when compared to the definition of the business or other issue described in the text record description. This allows the user to quickly scan through the results and determine which terms may be used for comparison, analytical or other purposes.

An innovation in the HCP is that the results can be forwarded to the Machine Learning platform as a labelled dataset where classification algorithms are used to further refine the HCP ability to classify text appearing in the text records to be analyzed. This combination of a rules-based system with Machine Learning greatly enhances the efficiency of the system.

Speed and accuracy are essential in decision making. The GUI is designed to allow the user to eliminate categories by selecting a displayed potential match then selecting the “delete” feature. All choices in that category are removed, clearing the viewing field. In addition, if the reviewer needs an in-depth view of a text record description, the reviewer can “click” on a recommended definition. When available, the definition will appear in the display window to assist the user in further processing.

In an additional embodiment, the HDP augmentation system may be deployed for utilizing machine learning. The HDP provides a multifaceted approach to utilizing machine learning that makes use of a feedback loop based on a rules-based system to improve the specificity and context of returns generated by the machine learning algorithms. The concept is that the use of dictionaries supplemented with the thesaurus feedback tool isolates facts and/or content of relevance. The identified facts and/or content become the training data for the machine learning algorithms.

This is especially useful for determining the prioritization of business decisions. The system can “read” the history, situations, actions undertaken, and other actions recorded from the business record fields in the business records as well as extract the same from the description from the observations within the text from the business records. Using this data, the system links to sources for business decision, management, and other resources available to the system. The system then uses natural language processing to compare the content of the business record with the data in the abstract. Similarities between the articles and one or more business decision analysis queries are presented to the reviewer as evidence for either refuting or supporting the course of action, and prioritization of courses of action, for solving one or more business issues, or recording such solutions in the data archives of the system.

The table can be selected from the dropdown list and the field to be analyzed from the dropdown list. The output of the analysis will be saved to a file named by the user once the create button is selected.

This triggers a new dropdown where the selected text is displayed in the window and the machine begins the analysis algorithm. The results are displayed in a table where the requested text is displayed. Matched terms are displayed as is the Specificity Score. The user can either accept the return using the Select box or remove the Section. In addition, the terms are highlighted according to matching definitions for MeSH terms, modifiers, and descriptors derived from pre-configured definitions and a MeSH Lexicon.

If the returns do not match or alternative terms need to be searched the key word search function can be deployed. Once selected, data is written to the file specified and can be further processed by the pipeline.

These data are now joined with business record data via the FHIR importer using the pipeline. Using the dashboard tools, select text is matched using the NLP tools. Based on these results, the user selects an autoconfigured web crawler for reference data to discover applicable text information. The data is processed by the data pipeline using the same configuration used for the business record data.

A principal innovation of the system is the ability to perform analytics without the need for programmers or developers.

The UI provides the user with access to the FHIR data acquisition system. The user then connects to an appropriate business record system using the URL name provided by the user or system administrator. This will auto-populate the FHIR resource fields in. An FHIR ape resource will be auto generated. The user can then preview the data that will be brought into the system by selecting the “Preview Data” button. If satisfied, the data will enter the system by selecting the “Create Table” button. These tables may now be placed in a Dashboard, be processed in ML algorithms, or undergo NLP analysis with subsequent Dashboard generation.

The list of data or “fields” is not limited to those displayed but rather serves as an example of the data types available for analysis. Any field present in the FHIR-compliant system can be captured by the system.

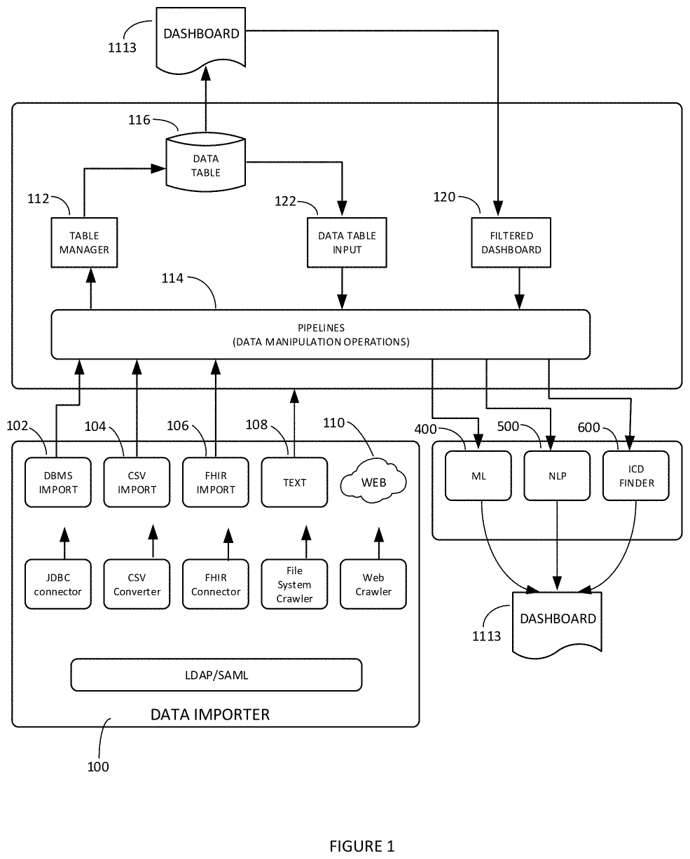

Turning now to FIG. 1, this figure presents a view of an Intelligence Augmentation System (IAS) features consistent with certain embodiments of the present invention. In an exemplary embodiment, the IAS accesses data from a number of online and network connected data repositories to import the data into the system for processing and analysis. In non-limiting examples, data may be sourced from the web 100 through the use of a web crawler 102, access data from text documents 104 through the use of a text document crawler 106, access data from relational database files 108 through the use of a database connector 110 with permission from the owner of the database files 108, and access comma separated value (csv) database files 112 through the use of a csv converter 114, again with the permission of the database file owner. This list of data sources may in no way be considered the only data sources from which the IAS may derive input data for analysis and processing. Additional data sources may be accessed through the use of additional data access methods.

In an embodiment, the incoming data from all data sources may be normalized and processed to be added to one or more data stores 116. A data store may be selected by a user for text processing and analysis 118 to discover textual data that conforms to one or more conditions expressed by a user for analysis. The data in the data store may also be accessed for quantitative analysis 120 and processed for decision support 122, again based upon parameters input and established by a user. After processing by any or all methods is complete, the processed data from the data store may be formatted for visual presentation 124 to the user.

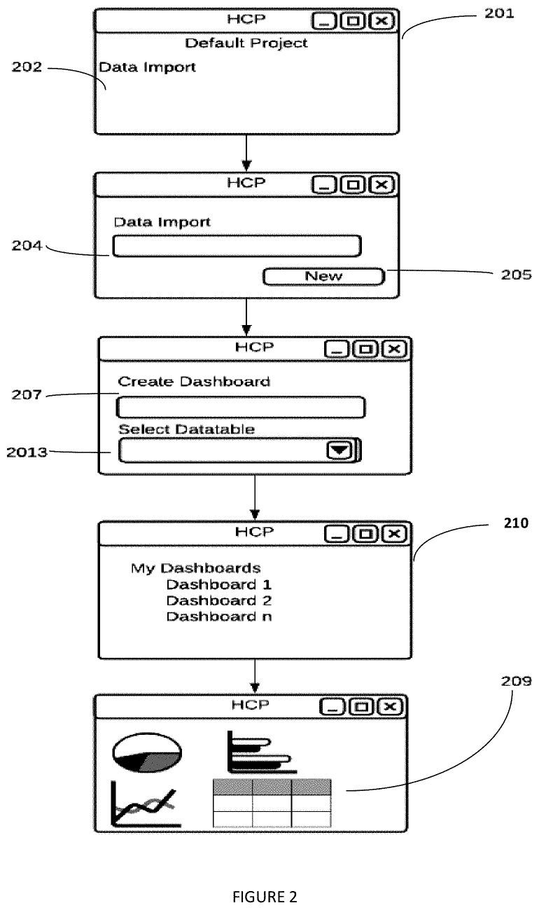

Turning now to FIG. 2, this figure presents a view of the IAS system configuration consistent with certain embodiments of the present invention. In an exemplary embodiment, the system presents a novel method to overcome the need for programming, the system user interface 200 is based on the NoviSystem advanced data modeling system (ADMS), consisting of a high-level programming function utilizing an object reference model that translates the criteria of data analysis established by the user into automatically generated processing steps in the form of SQL commands. This innovation results in the generation of a data table 202 that becomes the source of data for analytical queries and/or further data processing. The use of the ADMS provides flexibility in user functionality. Queries do not need to be designed to be domain specific. Rather, the model can be adapted to the data set that is being imported 204 regardless of whether the data was imported from formats such as text, csv records, database records, or any other pre-established data file format. New data 205 may be attached as generated in various pre-established data file formats. Furthermore, while a classic static database query system may require predefined primary and foreign keys to be maintained and may limit the ability to fuse multiple data sources, this approach allows disparate data types to be joined. The data generated as the new Data Table 202 is stored in a relational database 2013. The system may present a create Dashboard 207 option to a user permitting a user to select database tables to be presented in a Dashboard 207 view to a user. The Dashboard view 210 may present the user with a choice of Dashboards to be displayed. If a Dashboard is selected, it can be configured with data widgets 209.

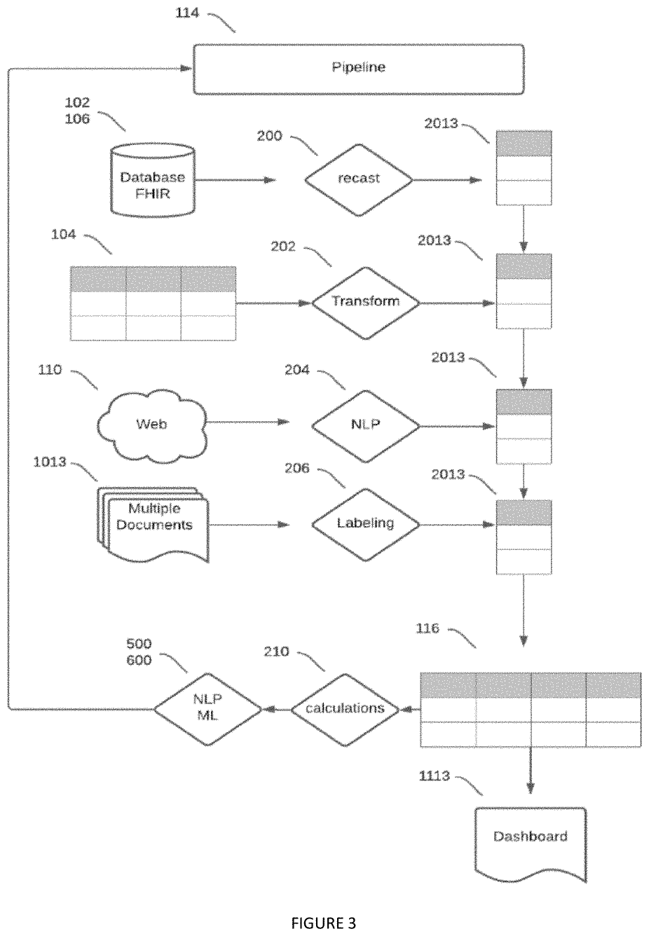

Turning now to FIG. 3, this figure presents a flow diagram for data import into the system consistent with certain embodiments of the present invention. The data pipeline 114 performs a series of high-level compute functions. In an exemplary embodiment, the system presents a novel method to overcome the need for programming, the system user interface 200 is based on the NoviSystem advanced data modeling system ADMS, consisting of a high-level programming function utilizing an object reference model that translates predefined SQL commands into automatically generated processing steps that meet the criteria of data analysis established by a user. This innovation results in the generation of a data table 116 that becomes the source of data for analytical queries and/or further data processing. The use of the ADMS provides flexibility in user functionality. Queries do not need to be designed to be domain specific. Rather, the model can be adapted to the data set that is being imported regardless of whether the data imported is formatted as text, csv records, database records, or any other pre-established data file format. Furthermore, while classic static database query systems may require predefined primary and foreign keys to be maintained and limit the ability to fuse multiple data source, this approach allows disparate data types to be joined. The data generated as the new Data Table 116 is stored in a relational database.

In an embodiment, the tasks performed by the pipeline are defined as follows: data may be imported from a variety of sources such as Databases 102, FHIR APIs 106, csv files 104, or the Web 110. The system, using a GUI queries the user regarding how data should be processed. This includes but is not limited to recasting 200, transformation 202, pre-processing for natural language processing 204, labelling or any combination thereof 206. Units from individual tables 208 can be recombined to form new tables 116 that can now undergo further quantitative analysis 210, machine learning 500 or natural language processing 600.

In this embodiment, resultant Dashboards 1113 are generated by the user using a dropdown configuration menu. The HDP has a GUI that allows non-programmers to develop queries of structured and unstructured data processed by the HDP algorithms.

The system may use a series of drop-down menus to direct the user to add data analysis functions to the display using screen position as a guide to where banners place query activities as rows across the top of a page while columns allow the user to configures the display into any number of columns. Each column may contain a separate analytic widget 2013.

The selection of Natural Language Processing 500 on the project page provides the functionality for implementing Natural Language Processing. Natural Language Process workflow offers the user two choices, a rules-based system using dictionaries 501, or machine learning 600. In a rules-based system, the system is directed by the user to annotate the document using the dictionaries developed using the Dictionary Editor 501. The advantage of a rules-based system is that the system will only annotate what has been defined as a term of interest, this term of interest becomes a dictionary term.

The Natural Language Process rules-based system can readily adapt to other lexicons provided to the system. Definitions from Healthcare/LifeSciences groups such as the National Center of Bioinformatic that contain dictionaries or lexicon can be imported into the system for use in the system, improving the specificity and context of search results tailored to the needs of the user.

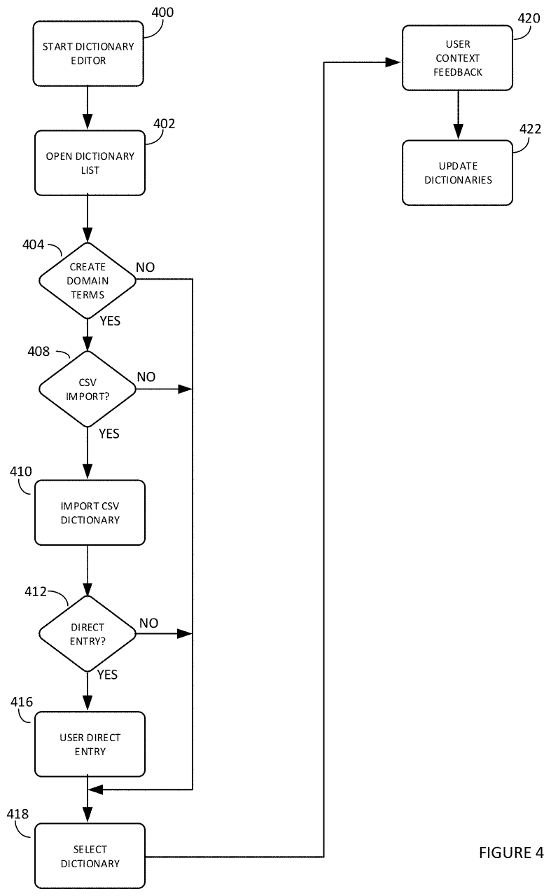

Turning now to FIG. 4, this figure presents a flow diagram for building and/or updating one or more dictionaries for use by the system consistent with certain embodiments of the present invention. In an exemplary embodiment, the Text tool system begins by the user selecting the Dictionary Editor 400 on the GUI Project page. This opens a listing of the dictionaries available in the application 402. A dictionary is a collection of terms that have a similar meaning, for example, disease would use a dictionary of terms associated with “disease” such as sick, ill, illness, etc. The user can create a new dictionary 402 by requesting and utilizing domain terms of importance to the user 404. The system also may inquire of the user at 408 whether the system is to import a list of terms as a csv file. If the user selects this option, the system may import a list of terms as a csv file 410. Selecting csv import opens a new window and that allows the user to browse the file system and select a preconstructed csv file containing terms of interest. Once selected, the file is imported. The user may also, alternatively or in conjunction with the imported csv file select direct entry of terms at 412. If the user selects the option to enter terms directly, the system provides a data entry capability to permit the user to enter the terms and/or words 412 in the spaces provided.

Dictionaries can be edited by selecting the dictionary in the GUI. The development of dictionaries can be a tedious process. To improve the efficiency of the process, selecting dictionary 416 provides the user with several options; viewing suggestions, view raw data, or delete.

Selecting suggestions initiates the thesaurus review process where the terms in the dictionary are compared to a thesaurus contained in the application. The synonyms, hyponyms, and hypernyms are then annotated in the data table along with the original dictionary terms. A sample of the sentences containing the original terms and synonyms and are presented to the user 418. The user can then review these sentences and determine if the context of the terms is appropriate and provide guidance as to appropriate terms as feedback to the system 420. If appropriate, the terms are added to the dictionary 422. The thesaurus process functions on textural data using a series of algorithms that are python-based but can be deployed using java.

The system has logic to filter user identified terms based on the text in a record being analyzed by the system. The filter may recognize demographic information such as the gender of the patient, as well as other demographic information that may negate gender-specific information for inclusion. The filter also handles anaphoras, disregarding terms presented such as “does not have” or “no sign of”, in a non-limiting example.

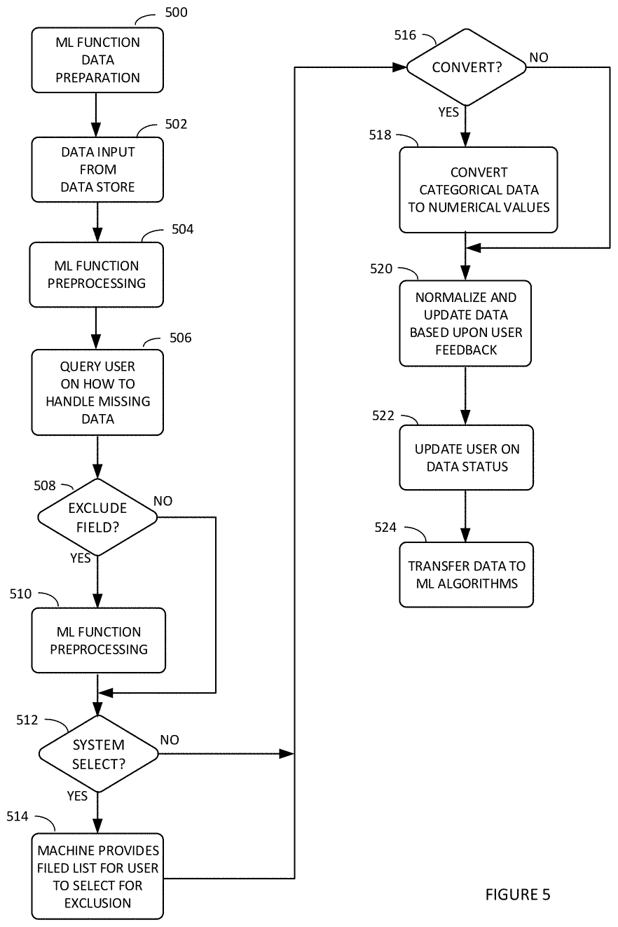

Turning now to FIG. 5, this figure presents a diagram that illustrates the functionality of the machine learning process. The drop-down menu system begins with preprocessing data 500 that has been selected from the data store 502. This includes statistical analysis of the data as well as determining data type as well as missing values 504. The user is then queried on how to handle missing data 506 and if classification of data type is correct 508. The system then queries the user for the performance of data set reduction algorithms 510 and presents results to the user for acceptance 512. The dataset is then further processed 516 and the user is asked if the finalized dataset needs to be reclassified 518. Once the response is given, the data is normalized 520 and the user is informed 522 that the data is ready for the machine learning algorithm 524.

The selection of the machine learning algorithms is another innovation of the HCP where the machine prompts the user for information then develops the protocols for tuning and testing various algorithms for accuracy and precision.

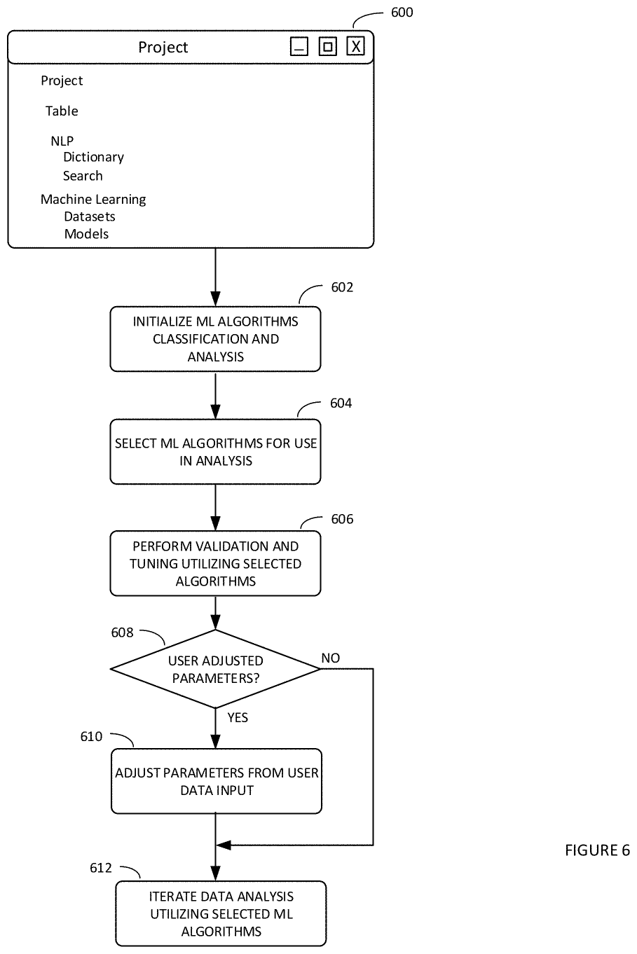

Turning now to FIG. 6, this figure presents an outline of the process for tuning and testing various algorithms for accuracy and precision. Once the datasets have been prepared, the user can select Models from Machine Learning on the Project UI 600. This initiates a series of pipeline activities 602, querying the user for the type of analysis that needs to be performed 604. Once input is received, the machine begins the internal process of splitting data into training and testing sets then performing cross-validation testing and performing the tuning of the algorithm by utilizing selected algorithms for cross testing 606. If necessary, the system will up-sample data to improve performance. A comparison is performed to determine if tuning parameters should be adjusted 608. Once complete, the accuracy and precision will be presented to the user along with the chance to alter parameters 610. Once user input is received, the model is developed and the data analysis is iterated through all incoming data records utilizing selected ML algorithms 612.

The types of models deployed by the system include regression, support vector machines, decision trees, ensemble methods, distance relationships(vectors), neural networks and their variants. The design of the system allows any machine algorithm to be deployed that accepts data that can be formatted into a table or array, therefore is essentially unconstrained.

To adapt to user preference for data display, the system uses a series of drop-down menus to direct the user to add data analysis functions to the display using screen position as a guide where banners place query activities as rows across the top of a page while columns allow the user to configure the display into any number of columns. Each column may contain a separate analytic widget.

The configuration of the data display is referred to as a dashboard. Each dashboard is associated with a primary data table in the data store. The system may either use established primary, foreign key relationships that exist in database tables or the system may generate these relationships in csv files or unrelated data tables imported into the HDP. Automatic dashboard generation increases the user's ability to assess relationships between data sets without the need of a programmer.

In an embodiment, the text tools herein described enable the user to develop models for fact extraction and text classification without a deep understanding of programming. This allows the HCP to extract a wide range of healthcare related facts depending on the knowledge domain of the user. The system relies on the user's expertise in the field to initiate the process and provides feedback to develop models for data extraction and text classification. The system is agnostic and can be used by any subject matter expert.

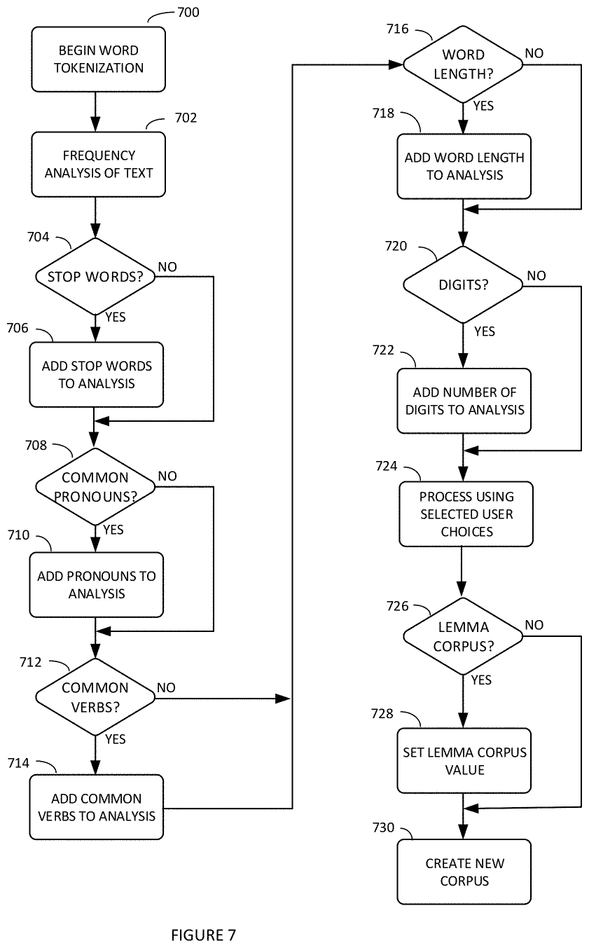

Turning now to FIG. 7, this figure presents a flow diagram for word tokenization and analysis consistent with certain embodiments of the present invention. In this embodiment, the system begins with processing text fields to tokenize words in any imported Data Table. The objective of the text cleaning process is to reduce the number of irrelevant words, terms that have no impact on context or specificity, so that the data set is reduced in size leading to more efficient operation and a greater probability of relevant returns.

The first step in the process is word tokenization 700. This breaks down the structure of the text data from continuous strings to individual tokens. When tokenization is complete the system performs frequency analysis 702 of the tokenized text using nitk or other suitable programming tools. This frequency value for each tokenized word may be stored for later use.

At 704, the system asks if stop words should be included in the analysis. If the user indicates that they should, stop words are included in the analysis by comparing word frequency values to stop word frequency at 706. The user is also presented with choices by the system to include common pronoun frequency at 708 and common verb frequency at 712. If the user elects to include common pronouns and common verbs in the analysis, common pronouns are added to the analysis at 710 and common verbs are added to the analysis at 714.

Two additional cleaning steps may be performed if selected. At 716 the user is asked if word length should be included, and, if elected by the user, the system removes any word less than four letters long with the exception of abbreviations at 718. At 720 the user is asked if digits should be removed and, if elected by the user, the system removes a selected number digits from the analysis at 722. The system processes the Data Table utilizing the user specified selections at 724 to create a new corpus. At 726 the system asks the user if the new corpus should be created using the lemma. If the user elects to create a lemma corpus, at 728 the system sets the lemma corpus value, and the new corpus, regardless of type, is created as the basis corpus at 730 and can then be used as the basis for machine learning.

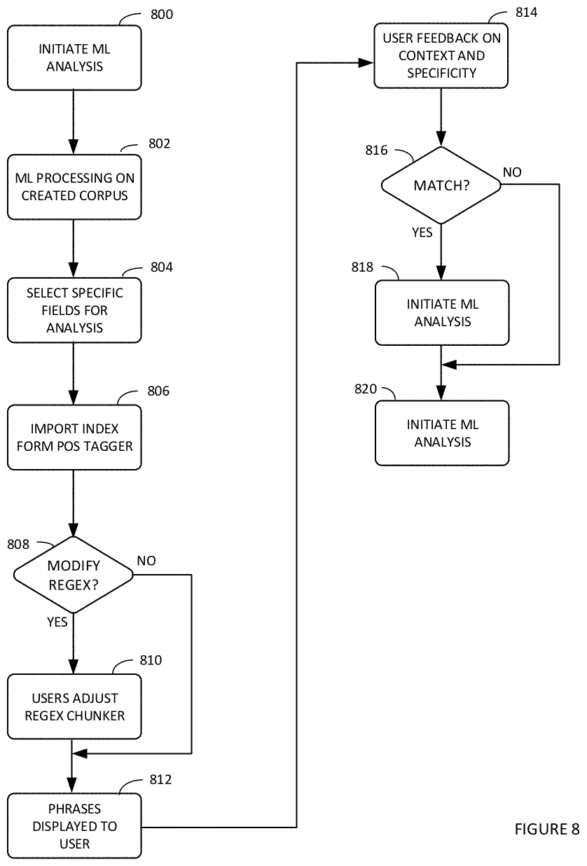

Turning now to FIG. 8, this figure presents a flow diagram for machine language preprocessing to build training data sets consistent with certain embodiments of the present invention. In this embodiment, the system initiates ML analysis at 800 by performing preprocessing steps on the previously created corpus at 802. The system selects specific fields for analysis at 804 and imports the necessary index from a POS tagger at 806. The system then ingests specific fields of cleaned text and the index from the POS tagger. At 808 the system inquires if the user wants to modify the regex. If the user selects this option, at 810 phrases are then generated using a regular expression chunker nitk or similar algorithm. The system has a default regular expression chunker but it can be adjusted by the user. Phrases are displayed to the user at 812 in order to receive user feedback on specificity and context at 814.

Following acceptance of the phrases, the POS tagging process is performed on either the lemma derived corpus or basis corpus. Terms from the phrases are compared to terms in the dictionaries for matching values at 816. One term from any dictionary must be present in a phrase. If there is a match, the phrase will be added to the training data at 818. At 820, the system updates the corpus and the updated corpus may be used in the machine learning algorithm for training.

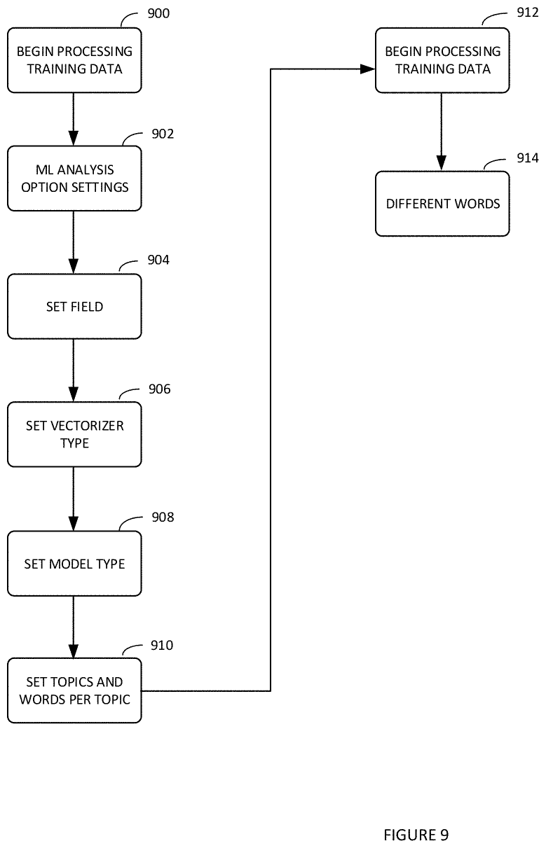

Turning now to FIG. 9, this figure presents a flow diagram for training data processing and use consistent with certain embodiments of the present invention. In this embodiment, the HDP uses multiple machine learning algorithms to process training data. The system may use a number of algorithms including but not limited to Latent Dirichlet

Allocation LDA, Non-Negative Matrix Factorization NMF, and Neural Networks NN. Machine Learning begins with processing the training data 900.

Users of the HDP are instructed to select analysis options from the user interface 902. The user may select the field to be analyzed at 904 and the vectorizer type may be selected at 906. Vectorization converts the text to a numerical array for use in the machine learning algorithms. The vectorizer type can either be a word to vector transformation or term frequency -inverse document frequency vectorization.

Following vectorization, the model type may be selected by the user at 908. This determines the clustering algorithm that will be run. The selection includes LDA, NMF, and NN as described above. At 910, the user may select the number of topics and the words per topic to be processed by the system. In a non-limiting example, the number of topics represents the number of clusters or topics that will be isolated by the machine learning algorithm. If the user asks for three topics, the returns will provide a list of terms in clusters that represent terms that cluster in three separate groupings.

This list is compared to the dictionaries and new terms or topics are presented to the user 912. The user can then elect to add the terms to a new dictionary or append the terms to an existing dictionary 914.

The combination of NLP and ML with the ability to “read” data records such as, in a non-limiting example, business records without the need of a data scientist represents a novel application and extension of the patent application “An Intelligence Augmentation System for Data Analysis and Decision Making” Docket Number: NOV-npr-001.

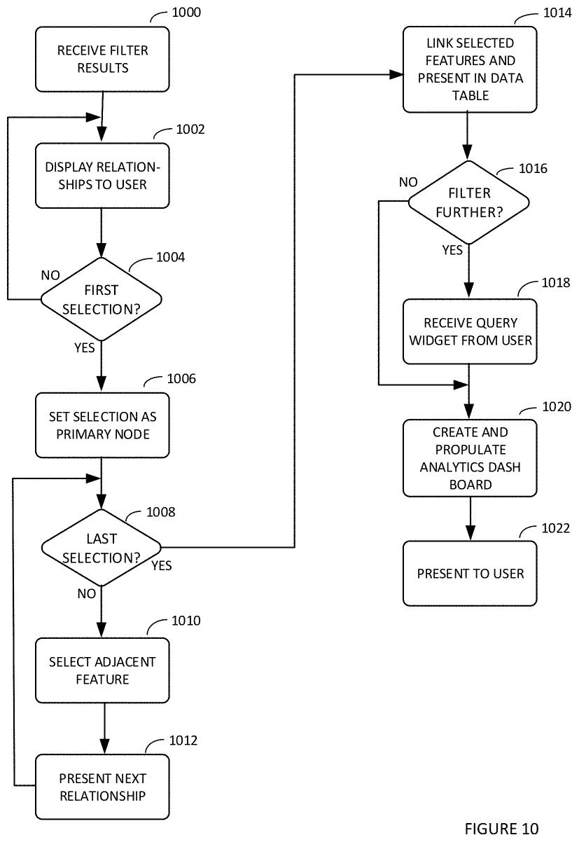

Turning now to FIG. 10, this figure presents a flow diagram for process for the creation of a knowledge graph consistent with certain embodiments of the present invention. The creation of a knowledge graph is initiated by receiving results from the filtering of data and derived data relationships as guided by the user queries and constraints at 1000. The system may then display the derived relationships to the user at 1002. The user is provided with a data relationship for selection at 1004. If the user does not select the provided data relationship as a first, or primary, selection for base relationship against which other selected data relationships may be visualized and/or creating the display of distance relationships between entities, the system may present a different relationship for the user's selection at 1002.

If the user selects the provided derived data term, this term will be utilized as the primary node, which is the initiation point of the selected data relationships that may be visualized at 1006. At 1008 the system checks to determine if this is the last data selection of the user. If it is not the last selection, the system presents other data relationships to the user for the selection of adjacent data features at 1010. The user is then presented with other data relationships for selection at 1012. If the user has chosen their final data relationship as presented from the system at 1008, the system may proceed to identifying and creating a visual representation of the data relationships by linking the selected features and creating the visualized data relationships in a data table at 1014.

At 1016 the user is provided with the opportunity to present additional filtering criteria for the data relationship display. If the user chooses to further filter the data and relationships presented, the system provides the user with the opportunity to create a query widget. The user may then use the query widget to provide additional filtering criteria at 1018, which are then transmitted to the system and used in additional data relationship filtering prior to the creation of a visual display of the data relationships. At 1020 the system utilizes all selected data relationships and any additional filtering criteria to create and populate a visual analytics dashboard. The completed visual analytics dashboard is presented to the user on a visual display device at 1022 without the need to engage a programmer or have support of programming assistance.

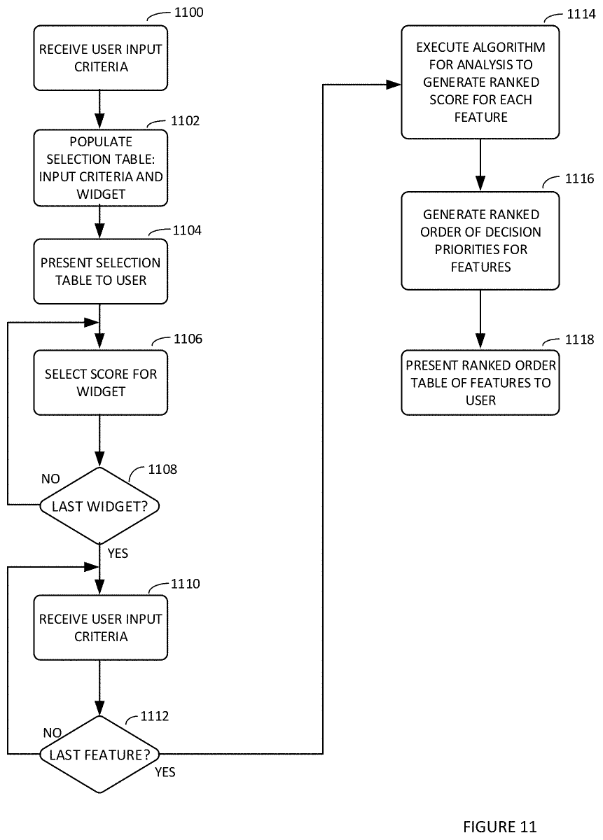

Turning now to FIG. 11, this figure presents a flow diagram for weighted order decision making consistent with certain embodiments of the present invention. At 1100 the system may receive user input specifying the criteria preferred by the user for the importance and order of data to be considered when making a decision. At 1102 the system may self-populate a selection table where the table is created with features that may be selected by a system widget that contains all of the criteria of importance to a user. At 1104 the system may present to a user the populated selection table through a user interface provided by the system. The user at 1106 may enter a score for each widget until the final widget score is entered at 1108. The system may then request the user input the relative weights associated with each of the features at 1110 until the last feature relative weighting is entered at 1112.

At 1114 the system utilizes the input scores and relative weights as input to an analytical algorithm. The system may then execute the algorithm for analysis to generate a ranked score for each feature. At 1116 the system may generate the ranked order of decision priorities utilizing the completed ranked scores. At 1118 the system may then present the ranked order table of features to the user to predict the decision making priorities and recommend the order in which the ranked priorities should be utilized to assist in the decisions that are being made by the user.

While certain illustrative embodiments have been described, it is evident that many alternatives, modifications, permutations and variations will become apparent to those skilled in the art in light of the foregoing description.

We claim:

1. A system comprising:
a data processor;
said data processor in communication with a display device enabled to display an analytics dashboard display;
said data processor receiving filtered data comprising one or more features of a business decision record;
said data processor providing said filtered data as input to an analytical algorithm instantiated within said data processor;
said data processor receiving ranked recommended order for business decision records resulting from analysis performed by said analytical algorithm;
said data processor creating a knowledge graph and a ranked recommended order record display and presenting said knowledge graph and said ranked recommended order record display to a user on said analytics dashboard display;
said user reviewing said analytics dashboard display and implementing said recommendations in the ranked order supplied by said data processor.

2. The system of claim 1, further comprising a Graphical User Interface (GUI) enabled to display said analytics dashboard display and active to receive input entered in said analytics dashboard from one or more users.

3. The system of claim 2, further comprising performing operations utilizing said input from said analytics dashboard to further filter and analyze one or more business records without requiring additional programming effort.

4. The system of claim 1, further comprising presenting a selection table containing said ranked recommended order record display and receiving user input criteria for priority preferences from said user.

5. The system of claim 4, where said data processor enables an analytical algorithm to further refine an analysis of said ranked recommended order record utilizing said input priority preferences.

6. The system of claim 5, where said data processor generates an updated ranked order of decision priorities and displays an updated ranked recommended order record within said analytics dashboard display.

7. The system of claim 1, where said ranked recommended order record display presents to a user a list of recommendations for the priority order in which business decisions are to be implemented.

8. The system of claim 1, where the ranking of the ranked recommended order record is performed based upon a scoring of the relative importance of each business decision contained within said ranked recommended order record.

9. The system of claim 1, further comprising utilizing one or more widgets where each widget further comprises a user generated score for relative importance of the widget.

10. The system of claim 1, further comprising a user entering a relative weight of importance for each feature contained within said ranked recommended order records.

11. A method, comprising:
displaying an analytics dashboard display on a display device;
receiving filtered data comprising one or more features of a business decision record;
providing said filtered data as input to an analytical algorithm instantiated within said data processor;
receiving ranked recommended order for business decision records resulting from analysis performed by said analytical algorithm;
creating a knowledge graph and a ranked recommended order record display and presenting said knowledge graph and said ranked recommended order record display to a user on said analytics dashboard display;
said user reviewing said analytics dashboard display and implementing said recommendations in the ranked order supplied by said data processor.

12. The system of claim 11, further comprising a Graphical User Interface (GUI) enabled to display said analytics dashboard display and active to receive input entered in said analytics dashboard from one or more users.

13. The system of claim 12, further comprising performing operations utilizing said input from said analytics dashboard to further filter and analyze one or more business records without requiring additional programming effort.

14. The system of claim 11, further comprising presenting a selection table containing said ranked recommended order record display and receiving user input criteria for priority preferences from said user.

15. The system of claim 14, where an analytical algorithm further refines an analysis of said ranked recommended order record utilizing said input priority preferences.

16. The system of claim 15, further comprising generating an updated ranked order of decision priorities and displaying an updated ranked recommended order record within said analytics dashboard display.

17. The system of claim 11, where said ranked recommended order record display presents to a user a list of recommendations for the priority order in which business decisions are to be implemented.

18. The system of claim 11, where the ranking of the ranked recommended order record is performed based upon a scoring of the relative importance of each business decision contained within said ranked recommended order record.

19. The system of claim 11, further comprising utilizing one or more widgets where each widget further comprises a user generated score for relative importance of the widget.

20. The system of claim 11, further comprising a user entering a relative weight of importance for each feature contained within said ranked recommended order records.
